(19) United States
(12) Patent Application Publication
(10) Pub. No.: US 2010/0170508 A1
(43) Pub. Date: Jul. 8, 2010

(54) NASAL DRUG DELIVERY DEVICE AND METHOD

(75) Inventors: Perry A. Genova, Chapel Hill, NC (US); Robert C. Williams, III, Raleigh, NC (US); Daniel Deaton, Apex, NC (US); Matthew Khare, Willow Spring, NC (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/724,160

(22) Filed: Mar. 15, 2010

Related U.S. Application Data
(63) Continuation of application No. 11/945,376, filed on Nov. 27, 2007, now abandoned.
(60) Provisional application No. 60/867,283, filed on Nov. 27, 2006.

(51) Int. Cl. A61M 15/08

(52) U.S. Cl. 128/203.12

(57) ABSTRACT

A nasal drug delivery device includes a pump that supplies drug containing fluid to a spray port. The pump is actuated by the user pressing on an externally accessible actuator. The actuation force from the user can be transmitted to the pump in a controlled fashion such that the pump sees a more consistent actuation force. Additionally or alternatively, the forward direction of the device's housing and/or the direction of actuator motion can be oriented with respect to the device's direction of spray so that the device can be conveniently held by a user for optimum results.

### CROSS-REFERENCE SECTION TO RELATED APPLICATIONS

This application is a continuation of U.S. patent application Ser. No. 11/945,376, filed on Nov. 27, 2007; which claims the benefit of priority to U.S. Provisional Patent Application No. 60/867,283, filed on Nov. 27, 2006, all of which are incorporated by reference in their entirety.

### FIELD OF THE INVENTION

The present invention relates to a nasal drug delivery device for delivery of liquid medicament to the nasal cavity, particularly the nasal epithelia.

### BACKGROUND OF THE INVENTION

Nasal delivery of pharmaceutical products can be useful both for treating diseases or disorders in the nasal passages themselves and for treating systemic and/or neurological disorders. However, it has been observed that particle or droplet size has significant impact on absorption when administering drugs via the nose and the nasal epithelia. Smaller droplets have been shown to impact on the higher nasal turbinates, which promotes better absorption into the body. On the other hand, droplets that are too small, and/or are delivered at too high a velocity, can be carried beyond the nasal passageway and undesirably find their way into the pulmonary region. Indeed, FDA Guidelines require testing to demonstrate that only a minimal amount of drug from a nasal delivery device be deposited beyond the nasal passageway and find its way into the pulmonary region.

Traditional devices for supplying drugs to the nasal epithelia include syringed nose drops, pump spray devices, and fluorinated propellant metered dose inhalers (MDI). These traditional devices have not generally been able to achieve the particle sizes necessary to maximize efficacy while helping mitigate undesired pulmonary absorption. For example, both eye dropper type devices and simple spray devices typically present medicament into the nasal cavity in a stream. The result is that much of the medicament simply runs out of the patient's nose, and only a small amount of the drug is absorbed, with even less of the drug reaching the nasal epithelia.

Newer pump type devices have increased ability to reduce the particle size of the medicament but have drawbacks of their own. Most pump devices rely on the user's hand strength to overcome a spring pressure in the pump, and create a pumping action. Typically, a significant force (e.g., about eight pounds) is required to actuate the device. While this presents little problem for some individuals, for many, particularly elderly and the young, the necessary force can be difficult to repeatably achieve. Moreover, many individuals end up with less than optimal sprays produced from such pumps because of the variation in action of applying the necessary power to the pump and/or the variability in hand strength. Other devices, known as metered dose propellant type devices, tend to produce good particle size, but at an undesirably high effective velocity. The pressure of the propellant in these devices tends to cause the drug to escape the nasal passageways and thus be deposited in the lungs or other portions of the pulmonary region.

Thus, there remains a need for alternative means of delivering a desired amount of drug to the nasal epithelia, advantageously in desired particle size distribution and/or at a desired velocity.

### SUMMARY OF THE INVENTION

A nasal drug delivery device according to the present invention includes a pump that supplies drug containing fluid to a spray nozzle. The pump is actuated by the user pressing on an externally accessible actuator. In some embodiments, the actuation force from the user is transmitted to the pump in a controlled fashion such that the pump sees a more consistent actuation force. In some embodiments, the device's housing and/or the direction of actuator motion are oriented with respect to the device's direction of spray so that the device can be conveniently held by a user for optimum results.

The nasal drug delivery device can include a housing having a spray port; storage container housing a liquidous medicament; a selectively actuable pump supported by the housing and operatively connecting the reservoir to the spray port; the pump having a plunger controlling operation thereof; an externally accessible actuator moveably coupled to the housing; and a first elastic element disposed operatively between the plunger and the actuator. Inward movement of the actuator to a first distance causes the elastic element to compress and thereby store energy. Inward movement of the actuator beyond the first distance causes the plunger to be depressed by the release of the energy stored in the elastic element to thereby force a portion of the medicament to be sprayed from the spray port. A catch can be associated with the plunger. The catch can resist inward movement of the plunger in response to inward movement of the actuator to the first distance, thereby allowing compression of the elastic element. The catch can move from a catch position to a release position in response to the actuator moving inward beyond the first distance to thereby release the plunger for inward movement under bias of the elastic element. Various embodiments of catch mechanisms are disclosed. A spray plume issuing from the spray port advantageously has a particle size distribution with a Dv50 value of approximately 50 um or less. Related methods are also disclosed.

In another embodiment, a method of administering a medicament nasally to a user includes: providing a nasal delivery device including: 1) a housing having a spray port; 2) storage container housing a liquidous medicament; 3) a manually powered pump supported by the housing and operatively connecting the reservoir to the spray port; 4) an externally accessible actuator moveably coupled to the housing; applying an actuation force of approximately four pounds or less to depress the actuator and thereby actuate the pump; and thereafter, delivering a portion of the medicament into the nasal passages of a user by generating a spray of medicament from the spray port having a particle size distribution with a Dv50 value of less than 50 um in response to the depressing of the actuator. The spray exiting the spray port can have a vertical flow. The method can further include compressing a spring associated with the device to store energy; and applying an actuation force can include thereafter releasing the stored energy, in response to the actuator being depressed beyond a first distance, to thereby actuate the pump.

In another embodiment, the nasal drug delivery device can include a housing extending in a first direction from a distal end portion to a proximal end portion with a spray port disposed proximate the proximal end; a reservoir operative to hold liquid medicament; a selectively actuable pump operatively connecting the reservoir to the spray port; wherein the spray port, which is advantageously configured to be inserted in a human user's nose, is oriented to spray medicament in a proximal second direction; the second direction forming a non-zero acute angle with the first direction. Thus, in some embodiments, the medicament sprayed from the spray port is directed away from the housing along a path that does not over-travel the housing. Related methods are also disclosed.

In another embodiment, the nasal medicament delivery device can include: a reservoir operative to hold liquid medicament; a housing including a spray port; the spray port configured to be inserted in a human user's nose and oriented to spray medicament in a first direction; a selectively actuable pump operatively connecting the reservoir to the spray port; an externally accessible actuator moveable relative to the housing in a second direction to actuate the pump and cause the medicament to be sprayed from the spray port; wherein a dot product of a first vector oriented in the first direction and a second vector oriented in the second (spray) direction is a non-zero positive value. The housing can have an upper surface and a lower surface, with the actuator disposed proximate the lower surface and the spray port proximate the upper surface. The pump can be a manually powered positive displacement pump. Related methods are also disclosed.

Other aspects of various embodiments of the inventive device and related methods are also disclosed in the following description. The various aspects can be used alone or in any combination, as is desired.

### BRIEF DESCRIPTION OF THE DRAWINGS

FIG. 1 shows a cross-sectional view of a nasal drug delivery device according to one embodiment of the present invention in ready configuration.

FIG. 2 shows a cross-sectional view of a device according to FIG. 1 in the initial stages of actuation, before movement of the catch arm to the release position, with the cover removed for clarity.

FIG. 3 shows a more detailed view of one portion of the device of FIG. 2.

FIG. 4 shows a cross-sectional view of a device according FIG. 2 beginning to release medicament just after the catch arm moves to the release position.

FIG. 5 shows a cross-sectional view of a device according to FIG. 2 ending the spray of medicament.

FIG. 6 is a graph and chart showing a typical prior art nasal pump particle size distribution at an actuation force of approximately five pounds.

FIG. 7 is a graph and chart showing a typical prior art nasal pump particle size distribution at an actuation force of approximately eight pounds.

FIG. 8 is a graph and chart showing a particle distribution at an actuation force of approximately four pounds for a device according to one embodiment of the present invention.

FIG. 9 shows particle sizes which can be produced by the device according to an embodiment of the present invention at selected actuation forces.

FIG. 10 shows a cross-sectional view of a nasal drug delivery device according to another embodiment of the present invention in ready configuration.

FIGS. 11A-B show a portion of the control mechanism of the device of FIG. 10, in side and front view respectively.

FIGS. 12A-B show another portion of the control mechanism of the device of FIG. 10, in side and front view respectively.

FIG. 13 shows a cross-sectional view of a device of FIG. 10 with the actuator button pressed to just before firing.

FIG. 14 shows a cross-sectional view of a device of FIG. 10 ending the spray of medicament.

### DETAILED DESCRIPTION

The present invention relates to a nasal drug delivery device 10 that includes a pump 50 that supplies drug containing fluid to a spray port 44. The pump 50 is actuated by the user pressing on an externally accessible actuator 80. In some embodiments, the actuation force from the user is transmitted to the pump 50 in a controlled fashion such that the pump 50 sees a more consistent actuation force. In other embodiments, the forward direction F of the device's housing 20 and/or the direction of actuator motion P are oriented with respect to the device's direction of spray S so that the device 10 can be conveniently held by a user for optimum results. These aspects of the invention can be used alone or in combination, as desired.

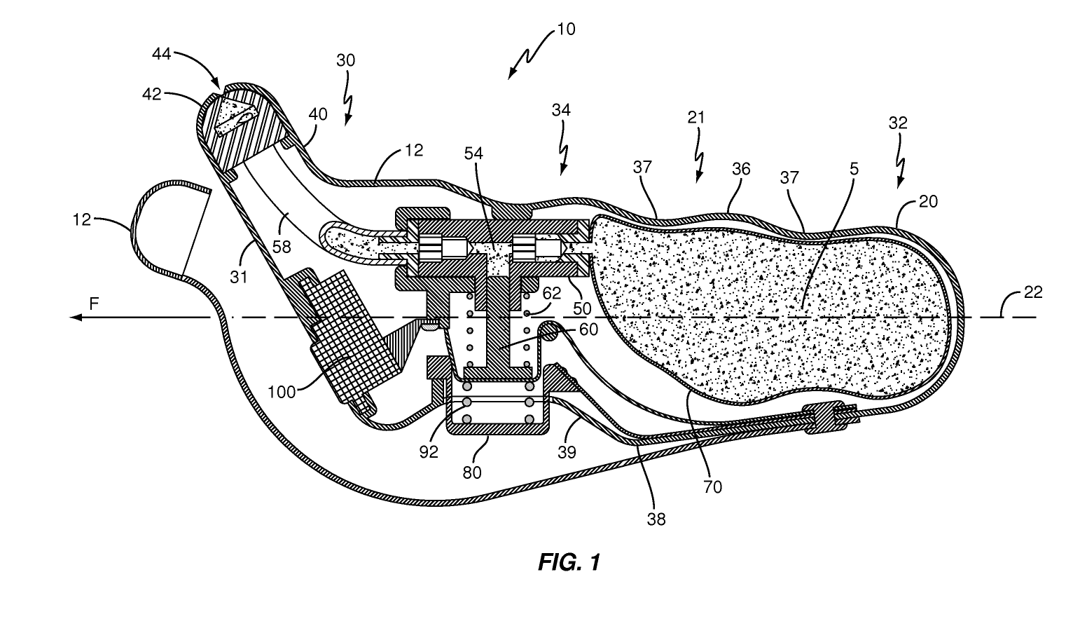

One embodiment of the nasal drug delivery device 10 is shown in FIG. 1. The device 10 includes a housing 20 and an actuator 80 moveably coupled to the housing 20. The housing 20 includes a proximal end portion 30, a distal end portion 32, and an intermediate portion 34. When held in the proper dispensing position by the user, the proximal end portion 30 is disposed closest to the user's face and the distal end portion 32 is disposed farthest from the user's face. Thus, for ease of reference, the direction from the distal end portion 32 to the proximal end portion 30 can be referred to herein as the forward direction F, with the opposite direction referred to as the rearward direction. For the embodiment shown in FIG. 1, the housing 20 is generally elongate along longitudinal axis 22, with a upwardly and forwardly extending protrusion 40 for the spray port 44, as discussed further below. Advantageously, the main portion 21 of the housing 20 is significantly longer along axis 22 than tall, and taller than wide, so as to aid the user in intuitively positioning the device 10 properly during use. The housing 20 can take a variety of forms, with the upper surface 36 of the housing 20 advantageously including a plurality of indentions 37 for accepting the user's fingers, and the lower surface 38 of the housing 20 advantageously including a suitable depression 39 for accepting actuator 80. The housing forward or proximal portion 30 includes a projection 40 that extends upwardly and forwardly. A spray port 44 is disposed in the tip portion 42 of this projection, and the tip portion 42 is intended to be inserted into the user's nose during use. As such, the tip portion 42 should be generally rounded and taper appropriately. The forward endface 31 of the housing 20 can be generally flat and extend parallel to the direction of projection 40. The housing rear or distal portion 32 can be configured as desired, with a rounded contour believed to be advantageous. If desired, an optional flexible cover 12 can be secured to the housing 20 on one end, with the other end of cover 12 selectively covering the tip 42 of protrusion 40 so as to protect the spray port 44.

The housing 20 is preferably made of a rigid plastic material and houses elements of the device 10. For example, the fluid reservoir 70 is disposed in the distal portion 32, the pump 50 is disposed in the intermediate portion 34, and the spray port 44 is disposed in the proximal portion 30.

The reservoir 70 is located in the housing 20 for storage of the liquidous medicament 5. While not required in all embodiments, the reservoir 70 is advantageously formed of a flexible material, such as polyolefin or silicone, so that reservoir 70 can collapse under atmospheric pressure as the medicament 5 is dispensed. Further, while the reservoir 70 is advantageously permanently disposed fully internal to the housing 20, the reservoir 70 can alternatively be only partially disposed in housing 20, and/or can be removable therefrom, as is desired.

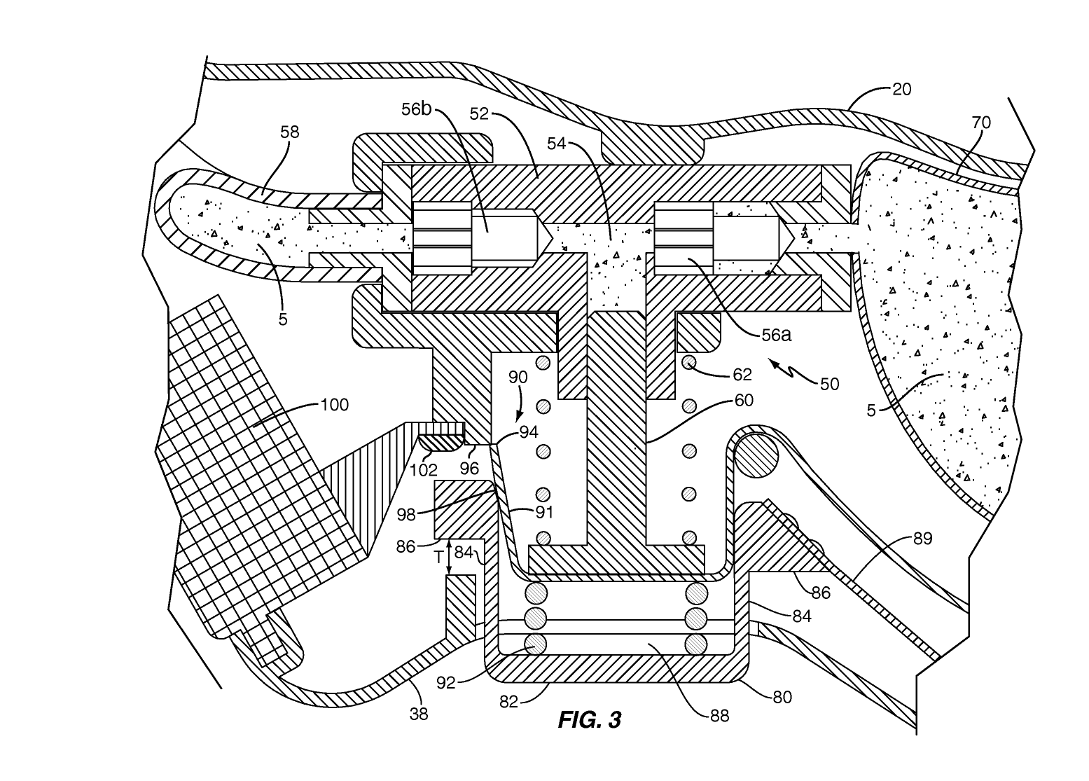

Pump 50 is operatively connected to reservoir 70 and acts to pump medicament from reservoir 70 to spray port 44 when actuated. The pump 50 can be of any type known in the art, but advantageously takes the form of positive displacement pump such as the elastomeric pump described in U.S. Pat. No. 6,223,746, the disclosure of which is incorporated herein by reference. In one embodiment, the pump 50 includes a main body 52 having a chamber 54, a pair of check valves 56a,56b, and a plunger 60. See FIG. 3. The check valves 56a,56b can be elastomeric check valves, ball and spring check valves, reed valves, or other check valves known to those of skill in the art. Inward movement of the plunger 60 toward chamber 54 causes medicament to be forced past check valve 56b and into the delivery channel (e.g., tube) 58 leading to spray port 44. The high pressure nature of this medicament supply causes the medicament 5 to be propelled through the delivery channel 58 and out spray port 44 in spray form. During this process check valve 56a prevents fluid flow back into reservoir 70. Following actuation of the pump 50, plunger 60 is released and begins to move away from the chamber 54, creating a vacuum in chamber 54. The vacuum in chamber 54 causes medicament 5 to flow from reservoir 70 through check valve 56a and into the chamber 54. Medicament 5 is prevented from flowing through check valve 56b because check valve 56b is in the closed position with the vacuum maintaining it as such. Medicament 5 is drawn into the chamber 54 until plunger 60 returns to its rest position. As can be seen, plunger 60 is acted upon by two opposing springs. Reset spring 62 resides between plunger 60 and chamber 54 and acts to urge plunger 60 outward to the ready position. Actuation spring 92 resides between actuator 80 and plunger 60 and is involved with actuation of plunger, as discussed further below.

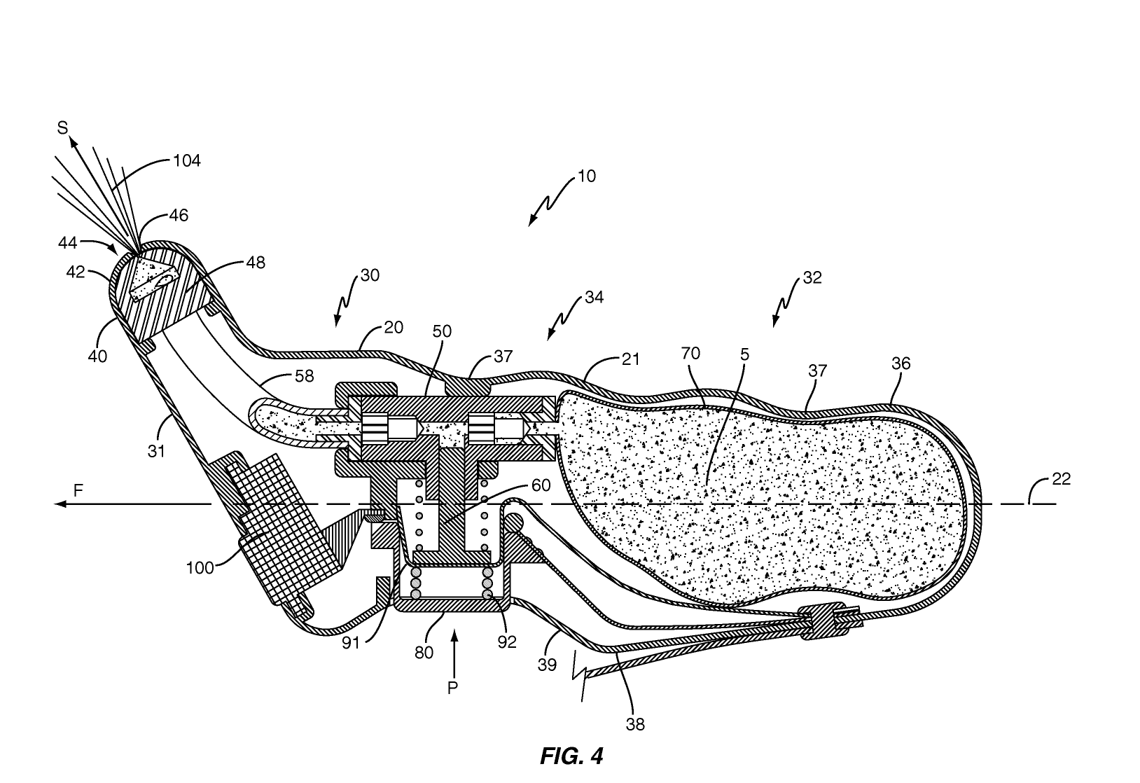

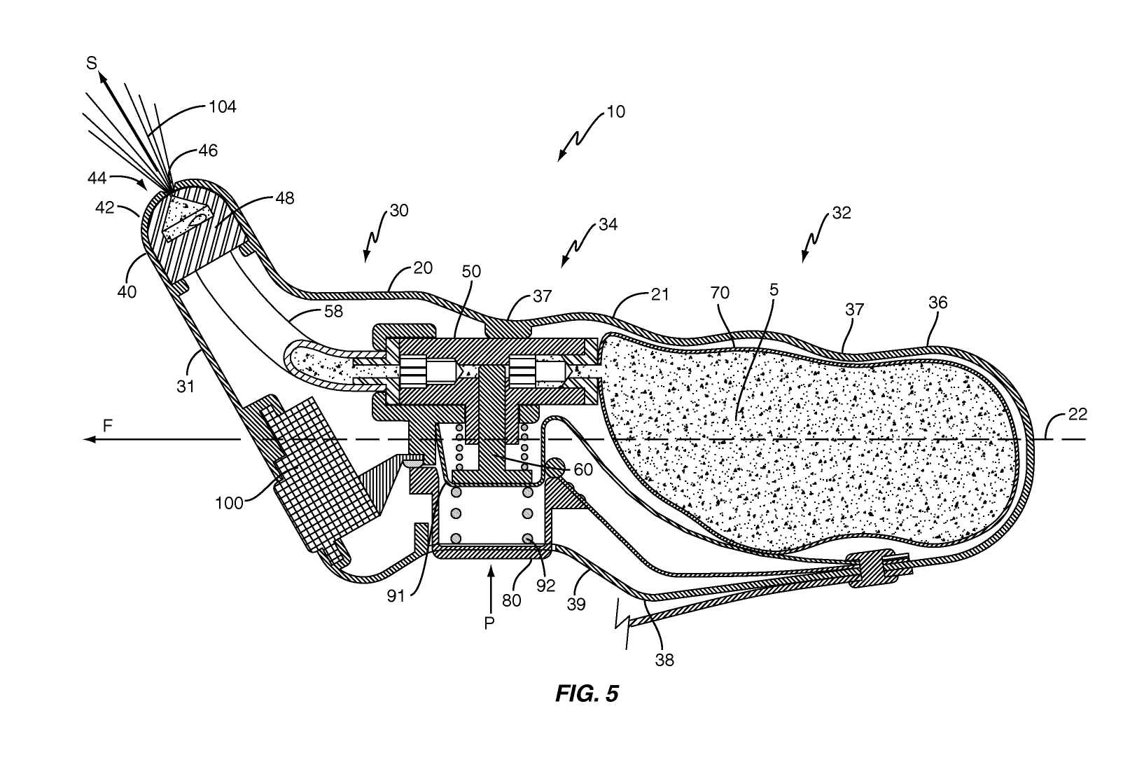

As shown in FIGS. 4-5, medicament 5 is expelled from the device 10 via spray port 44. The spray port 44 includes an opening 46 in housing 20 and a nozzle 48 disposed immediately upstream from opening 46. The opening 46 is disposed on tip 42 of protrusion 40 and is advantageously flared outward, such as by being tapered in a conical fashion. The nozzle 48 is mounted in housing 20 immediately behind the opening 46 and acts to atomize the medicament 5 into a fine mist. The nozzle 48 can take any form known in the art, but advantageously takes the form of a vortex nozzle, such as that described in U.S. Pat. No. 6,418,925, the disclosure of which is incorporated herein by reference. The spray output from nozzle 48 forms a plume 104, advantageously with a vertical flow; for purposes herein the functional midline of this plume 104 defines a spray direction S.

As can be seen in FIG. 4, this spray direction S for the embodiment of FIG. 1 is upward and outward, away from the main body 21 of housing 20, such that the spray plume 104 follows a path that does not travel back over the housing's main body 21. Thus, the included angle θ between a vector representing the spray direction S, and a vector oriented in the housing's forward direction F is a non-zero acute angle. Because of this, the dot product of these two vectors is a positive value. This relationship between the main housing body 21 and spray direction S allows the device 10 to be comfortably held in front of the user's face in an orientation that extends mainly directly away from the user's face, rather than vertically upward in front of the user's eye as in some prior art devices. Such ability is believed to encourage greater acceptance by potential users.

Referring to FIG. 3, actuator 80 typically takes the form of a simple button 80 that is moveably mounted to the housing 20. The button 80 is advantageously mounted in housing intermediate portion 34 proximate lower surface 38 so as be externally accessible. The button 80 can include a exterior user contact surface 82, inwardly extending legs 84, and an interior cavity 88. The exterior contact surface 82 can be contoured and/or textured as is appropriate. Each leg 84 can include a suitable flange 86 to help keep the button 80 secured to the housing 20. The button 80 is advantageously longitudinally secured with respect to housing 20, such as by being a relatively tight fit with respect to the corresponding opening in the housing 20 and/or by use of a retention element 89, so as to minimize lateral wobbling sensed by a user. Cavity 88 is defined by legs 84 and the portion of button 80 forming contact surface 82. Spring 92 rests in cavity 88 and tends to urge button 80 and plunger 60 away from each other.

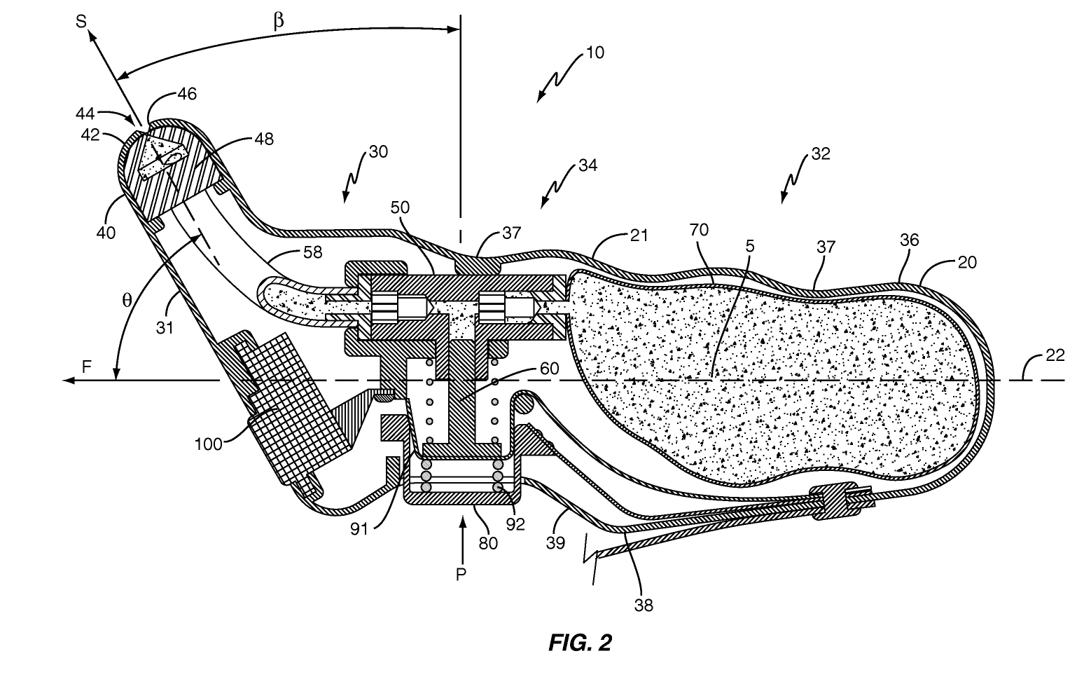

When pressed, actuator 80 moves in an actuation direction P inward toward pump 50. As can be seen in FIG. 2, a vector oriented in the actuation direction P is at a non-zero angle β to the spray direction S. In the embodiment of FIG. 2, the actuation direction P is directly up, while the spray direction S is up and to the left. Thus, the dot product of these two vectors is a positive value. Accordingly, pressing the actuator 80 in order to actuate the pump 50 applies a force that has a positive component in the spray direction S, meaning that such a force does not tend to pull the device 10 out away from the user's nose during actuation. Such an arrangement is believed to provide enhanced results in practice because there is less tendency to displace the spray port 44 from a desired location during actuation.

Pressing of actuator button 80 leads to activation of pump 50, resulting in the spraying of medicament 5 from spray port 44. However, because pump 50, in some embodiments, is a manually powered positive displacement pump, the force applied to plunger 60 affects the effective fluid pressure of the medicament 5 supplied to spray port 44 which, in turn, affects the particle size distribution of the resulting spray. As pointed out above, the applied actuation force tends to vary greatly from user to user and/or from actuation to actuation. To counter this, the force transmitted from actuator button 80 to the plunger 60 is controlled, in some embodiments, by a novel control mechanism 90. Referring to FIG. 3, one embodiment of the control mechanism 90 includes spring 92 and a catch arm 91 that interacts with a stop boss 96 and a cam surface 98. The catch arm 91 is a elongate body, typically formed of spring steel, that is secured to plunger 60 in cantilevered fashion. The catch arm 91 extends away from plunger 60 so that the arm's tip 94 is located in spaced relation to plunger 60. The catch arm 91 is intended to be deflected such that the arm's tip 94 can move between a hold position relatively farther from plunger 60 (FIG. 2) and a release position relatively closer to plunger 60 (FIGS. 4-5). The stop boss 96 can be formed on housing 20 or pump 50 and helps to support catch arm 91 against movement in the actuation direction. The cam surface 98 is associated with actuation button 80, such as being a portion of leg 84, and is located relatively closer to plunger 60 (in side view) than the location of catch arm tip 94 in the hold position. The movement of the cam surface 98 relative to catch arm 91 when button 80 is pushed helps urge catch arm 91 toward the release position, as discussed below.

Prior to the user pressing button 80, spring 92 is in a neutral or slightly compressed state and plunger 60 is urged outward by reset spring 62. When button 80 is initially pressed (FIG. 2), button 80 travels inward relative to housing 20; however, plunger 60 is prevented from moving inward because tip 94 of catch arm 91 abuts against stop boss 96. As such, pump 50 is not immediately activated. Thus, as a result of the user's pressing, button 80 is displaced toward plunger 60, compressing actuation spring 92 to effectively store the energy input by the user to depress actuator button 80. As displacement continues, cam surface 98 bears against catch arm 91 at locations that are farther and farther from where catch arm 91 mates with plunger 60. Because cam surface 98 is relatively closer to plunger 60, catch arm 91 is deflected inward by cam surface 98. When the button 80 has be pushed inward sufficiently, cam surface 98 deflects catch arm 91 sufficiently toward plunger 60 to displace catch arm tip 94 off of stop boss 96 and into the release position. When occurs, the catch arm 91 is no longer able to prevent the plunger 60 from moving toward pump 50, and the energy stored in spring 92 acts to press plunger 60 in toward pump 50, triggering the start of pumping action (FIG. 4). The amount of travel of the actuator 80 from the ready position until just before the cam surface 98 forces catch arm 91 to the release position is called the trigger distance T. Thus, depression of the actuator 80 beyond the trigger distance T triggers the pumping action. The actuator 80 can be further depressed during the pumping action until the actuator encounters a stop, such as stop boss 96. The pumping action continues as the energy from spring 92 pushes plunger 60 in toward chamber 54 until the plunger 60 reaches its full travel and/or the energy is expended. Of course, the actuation force required to trigger the pumping action is a function of the force balance between spring 62 and spring 92 as the button 80 is pressed. Advantageously, the actuation force is on the order of four pounds so as to be readily achieved by a wide variety of users, while being enough to provide a good tactile feel.

When button 80 is released after the pumping action, spring 92 urges button 80 away from plunger 60 and reset spring 62 urges plunger 60 away from pump main body 52. Movement of the plunger 60 away pump main body 52 has the effect of creating a vacuum in pump chamber 54, causing more medicament 5 to be “pulled” from reservoir 70 into pump chamber 54. When the plunger 60 has moved sufficiently, the catch arm 91 tip becomes free to move away from the plunger 60 to re-assume the hold position. The device 10 is then reset and ready for its next use.

From the above, it is clear that some embodiments of the device 10 utilize an indirectly actuated plunger 60 rather than a directly actuated plunger 60. Thus, the spray generation characteristics, while dependent on manual activation, become less determined by the magnitude of the actuation force applied to actuator button 80 by user, and more a function of the device itself. Accordingly, the resulting spray generation becomes more consistent. In particular, the plunger 60 is primarily actuated by energy stored in actuation spring 92, with the relative positions of the catch arm tip 94, cam surface 98, and stop boss 96, and the strength of spring 92, helping to determine the amount of energy stored in spring 92 at the moment of release. The amount of stored energy in spring 92, in turn, helps determine the actuation force applied to plunger 60, and thus the force of the pumping action of pump 50. And, the force of the pumping action helps determine both the velocity and particle size distribution of the spray exiting spray port 44. By using the controlled release approach described above, the actuation pressure applied to the plunger 60 is consistent and close to identical every time the plunger 60 is compressed. In addition, the release of the stored energy from the actuation spring 92 is nearly instantaneous, that is immediately after the catch arm tip 94 is moved off the stop boss 96. This results in a very consistent delivery of the medicament 5 from pump 50 to spray port 44, and therefore a more consistent generation of a medicament spray from device 10. Thus, a more controlled spray can be generated by the device 10 employing the novel control mechanism 90 under a broad range of forces applied to actuator button 80.

It should be noted that because the interaction between catch arm tip 94 and stop boss 96 helps control the amount of stored energy applied to plunger 60, it can be advantageous to control the alignment of the catch arm 91 relative to stop boss 96. For example, some embodiments can use a catch arm 91 that is relatively wide (in the direction into the plane of FIG. 1) so as to provide sufficient resistance to the movement of the plunger 60. However, such embodiments are somewhat vulnerable to miss-alignment between the catch arm 91 and the stop boss 96, causing a variability in the trigger distance T corresponding to the point at which the catch arm 91 moves from the hold position to the release position. To combat this, the catch arm 91 can be affixed to the plunger 60 at two or more spaced locations, and the plunger 60 can be provided with anti-rotation means, such as a rib/groove arrangement (not shown) with the pump main body 52. Because the catch arm 91 is rotationally fixed relative to the plunger 60, and the plunger 60 is rotationally fixed relative to the stop boss 96, the catch arm 91 is maintained in a more consistent alignment. In some embodiments, the catch arm 91 can extend from only one side of the plunger 60; in other embodiments, the catch arm 91 can extend across the plunger 60 and be secured to the housing 20, such as shown in FIG. 1.

The discussion above has assumed that the device 10 includes reset spring 62 and actuation spring 92 for applying their respective biases to plunger 60. However, it should be understood that any form of elastic element known in the art (e.g., compressible foam) could be used for the desired biasing action, and conventional coil springs are not required in all embodiments.

Further, it can be desirable to keep track of the number of actuations of pump 50. As such, some embodiments of the device 10 can include an optional dose counter 100. Any form of dose counter known in the art can be used, such as those described in U.S. Pat. Nos. 5,544,647 and 5,622,163, and U.S. patent application Ser. No. 10/625,359, the disclosures of which are incorporated herein by reference. Advantageously, the dose counter 100 is configured so as to be indexed by the sudden movement caused by the catch arm 91 moving from the hold position to the release position. For example, a portion of the button 80 can impact a contact 102 connected to the dose counter 100 to increment/decrement dose counter 100 in a conventional fashion. This contact 102, and/or the stop boss 96, can also act as positive stop for the actuator button 80, if desired. Other functionality can also be incorporated into the dose counter 102 using features known to those of skill in the art.

Several tests have been run in order to investigate the relationship between actuation force and spray characteristics, and to examine the effect of the controlled release pumping action described herein. In particular, analyses of particle size and particle size distribution were undertaken using the Dv50(um) setting on a Malvern® Spraytec particle size distribution device for various conditions discussed below.

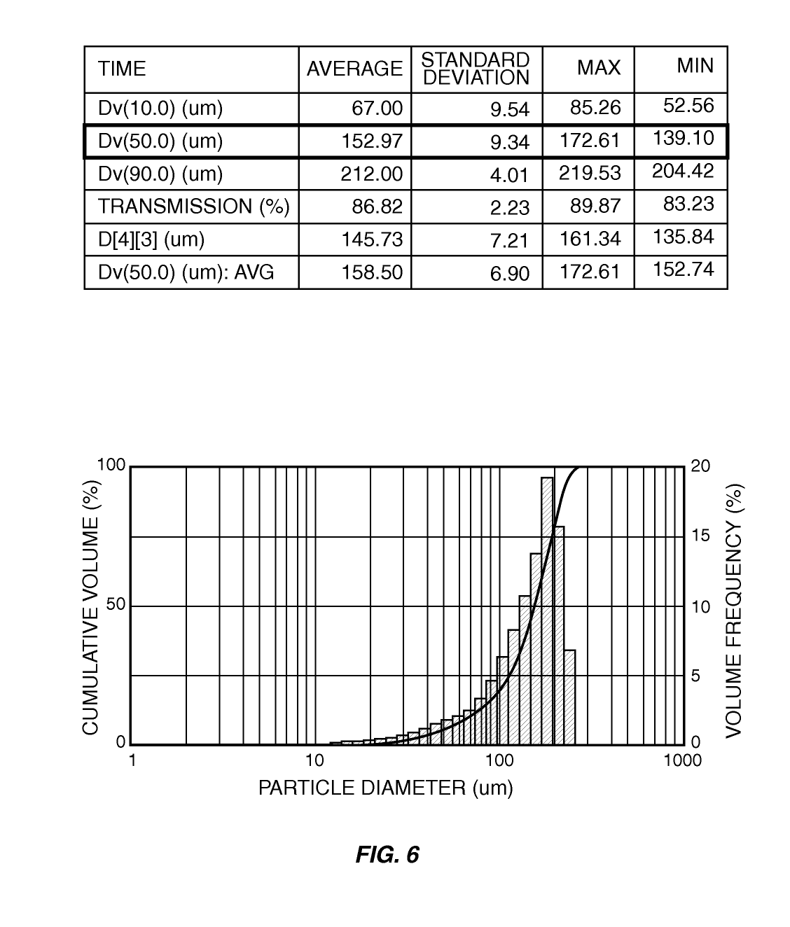

Test 1: a Rhinocort Aqua Nasal Pump was tested using a five pound actuation force, with the results shown in FIG. 6. As can be seen, the average particle size was approximately 153 um. This is well above a desired size of about 20-40 um which has been observed to produce good results for absorption of medicament through the nasal passages and the nasal turbinates.

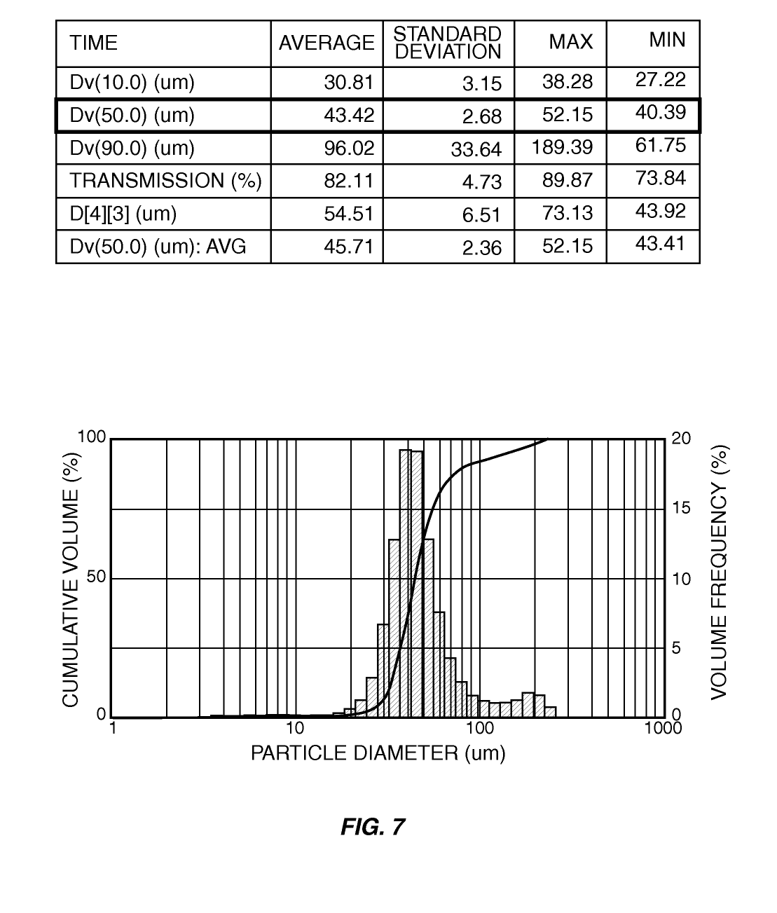

Test 2: a Rhinocort Aqua Nasal Pump was tested using an eight pound actuation force, with the results shown in FIG. 7. Though much better than at five pounds of force, the device still only produced an average particle size diameter of approximately 43 um. Moreover, this was only achieved at an eight pound force, which can be difficult for many users to achieve, and is sufficiently large that achieving consistent and repeatable results is a challenge.

As can be easily seen in a comparison of Test 1 and Test 2, the prior art experiences significant variation in spray characteristics at different actuation forces. In addition, the prior art approach is significantly challenged to produce a spray plume having the desired particle sizes and size distribution.

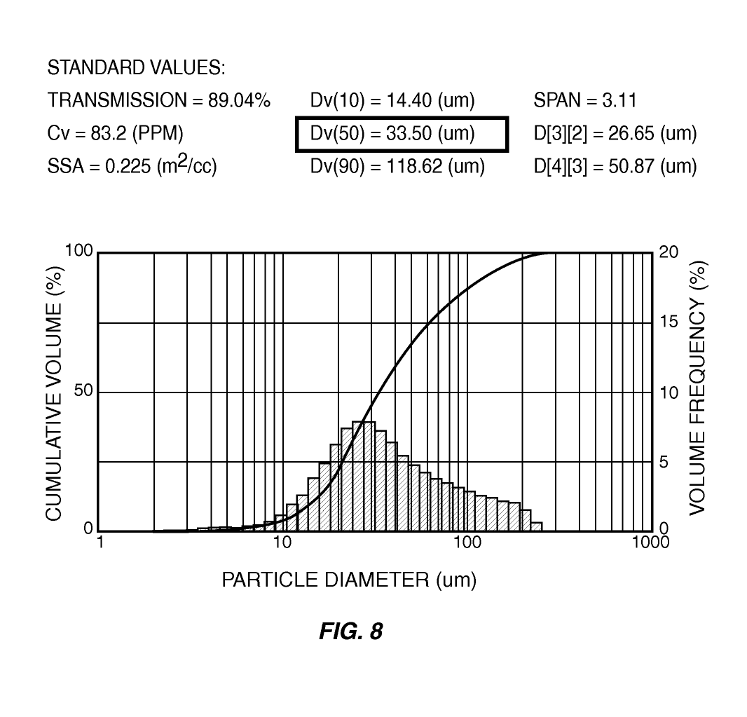

Test 3: a device 10 according to the present invention was tested using a four pound actuation force, with the results shown in FIG. 8. With an average particle size of only 34 um, and at less than half the force of the best results achieved by the prior art nasal pump device tested, the present nasal delivery device 10 achieved far superior results with a more manageable application of force.

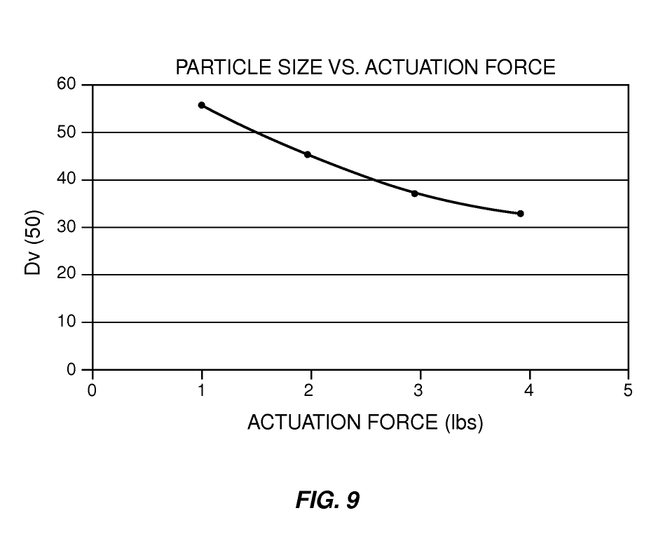

Further tests were also run on a device 10 according to the present invention at various activation force levels, with the results shown in FIG. 9. As can be seen, even at a low activation force of a single pound, the Dv50 particle size is about 55 um, which is far superior to the prior art device even at five pounds of activation force. And the results show relatively small variation in Dv50 particle size across the various activation force levels, suggesting a more robust design.

Accordingly, some embodiments the device of the present invention produce effective and repeatable doses of medicament to be applied to the nasal mucosa and turbinates with far superior average particle size, using lower applications of force, when compared with prior art devices. Moreover, the particle size of 20-40 um produced by such embodiments, though small enough to achieve rapid absorption in the nasal turbinates, is not so small that the medicament is readily transported past this region and into the pulmonary system.

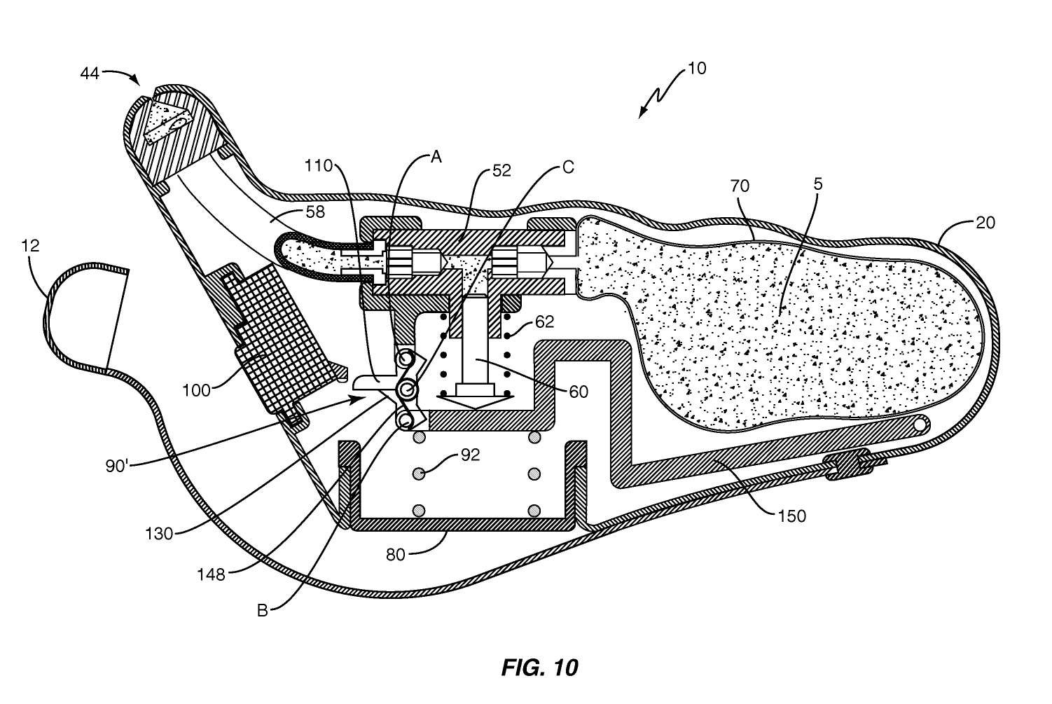

While FIG. 3 illustrates one embodiment of a control mechanism 90, other configurations of control mechanism can alternatively be used. For example, another embodiment of a control mechanism is shown in FIG. 10. The control mechanism 90′ of FIG. 10 is a form of a mechanism that includes a trigger arm 150 and a collapsible catch that includes primary pivot link 110, secondary pivot link 130, and bias spring 148. Trigger arm 150 is rotatably mounted to housing 20 at one end and is disposed between plunger 60 and actuation spring 92. While trigger arm 150 is shown as having four bends in FIG. 10, any suitable shape can be used for trigger arm 150.

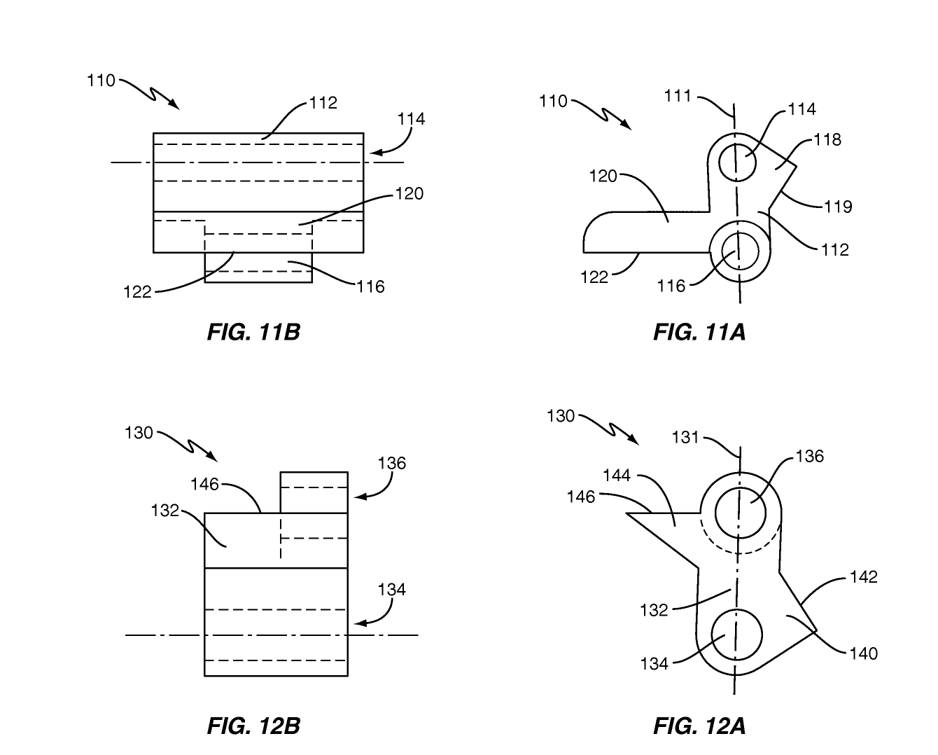

One embodiment of primary pivot link 110, shown in more detail in FIGS. 11A-B, includes a main portion 112 and an arm 120 extending therefrom. The main portion 112 includes a mounting pivot passage 114 at pivot point A and a joint pivot passage 116 at pivot point B. In addition, primary pivot link 110 includes a protrusion 118 extending generally opposite to arm 120. Protrusion 118 includes a stop face 119 disposed at an angle relative to longitudinal axis 111 running through pivot point A and pivot point B. The angle of the stop face 119 relative to axis 111 can advantageously be less than 45°, such as approximately 33°. The arm 120 extends out from main portion 112 generally perpendicular to axis 111. The arm 120 includes an abutment face 122 facing toward secondary pivot link 130.

One embodiment of secondary pivot link 130, shown in more detail in FIGS. 12A-B, includes a main portion 132 and a pair of protrusions 140,144. Main portion 132 includes a mounting pivot passage 134 at pivot point C and a joint pivot passage 136 at pivot point B. Protrusion 140 is disposed more distally from primary pivot link 110 and extends outward in one direction, while protrusion 144 is disposed more proximate primary pivot link 110 and extends in an opposite direction. Protrusion 140 includes a stop face 142 disposed at an angle to longitudinal axis 131 running through pivot point C and pivot point B. The angle of the stop face 142 relative to axis 131 can advantageously be less than 45°, such as approximately 33°. Likewise, protrusion 144 includes an abutment face 146 that extends generally perpendicular to axis 131 and is disposed to face toward primary pivot link 110.

Primary pivot link 110 is pivotally mounted to housing 20 at pivot point A using any suitable means (e.g., pivot pin, etc.). Secondary pivot link 130 is likewise pivotally mounted to trigger arm 150 at pivot point C. Primary pivot link 110 and secondary pivot link 130 are also pivotally joined together at pivot point B using any suitable method. Of course, the male/female relationship of the various parts can be reversed or otherwise altered, as is desired. Further, primary and secondary pivot links 110,130 can advantageously include suitable recesses so as to facilitate the connection and relative rotation thereof.

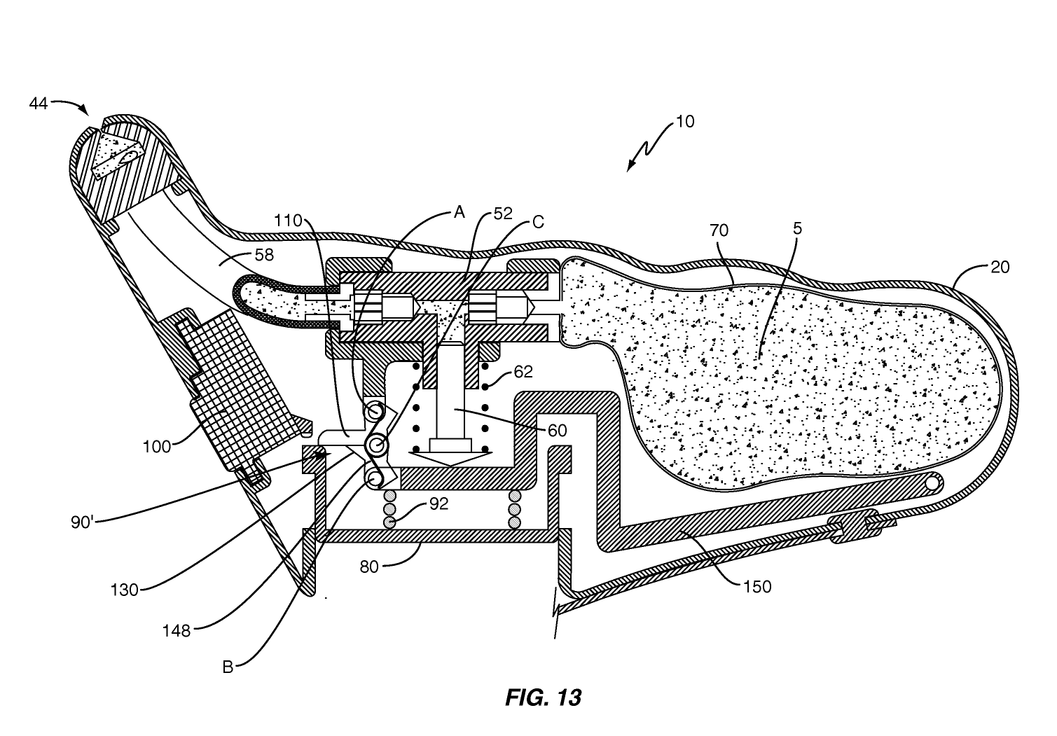

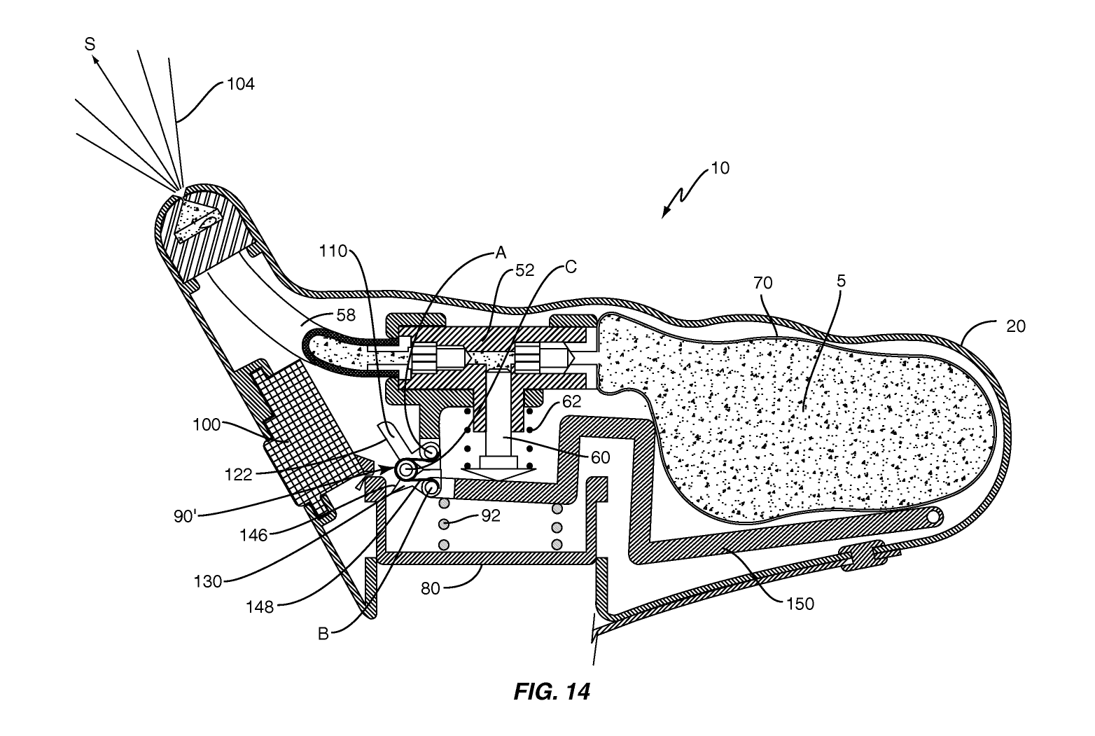

Referring to FIG. 10, the device 10 is shown before actuator button 80 is pressed to fire the device 10. At this point, actuation spring 92 urges button 80 outward, while reset spring 62 urges plunger 60 outward. As such, trigger arm 150 is urged away from pump main body 52 (counter-clockwise in the illustrations). In addition, spring 148 of control mechanism 90′ urges primary pivot link 110 and secondary pivot link 130 to a position (resist position) where pivot point B is to the right of a theoretical line between point A and point B. In this resist position, primary pivot link 110 and secondary pivot link 130 cooperate to resist clockwise rotation of trigger arm 150. Note that additional rightward rotation of the pivot links 110,130 is prevented by abutment faces 122,146 abutting against each other; thus, the rightward motion of point B is limited by the positive stop action of the interaction of abutment faces 122,144. When actuator button 80 is pressed, actuation spring 92 is compressed between actuator 80 and trigger arm 150. See FIG. 13. Trigger arm 150 is prevented from being displaced toward pump 50 by control mechanism 90′, specifically the relative orientations and interaction of primary pivot link 110 and secondary pivot link 130. After actuator button 80 has been pressed enough, actuator button 80 engages arm 120 on primary pivot link 110, and acts to rotate primary pivot link 110 clockwise. Rotation of primary pivot link 110 in this direction causes the pivot joint at point B to move leftward. Fairly quickly, point B will have moved leftward so as to now be on the left of theoretical line between pivot point A and pivot point C. Once this occurs, the primary and secondary pivot links 110,130 are free to rapidly move to a collapsed configuration (FIG. 14) and therefore no longer act to resist the clockwise (inward) movement of trigger arm 150. This releases trigger arm 150 to move suddenly toward pump main body 52, thereby depressing plunger 60 and resulting in spray being emitted from spray port 44. Clockwise movement of trigger arm 150 continues until stop faces 119,142 abut against each other so as to act as a positive stop to prevent over-depression of plunger 60 as shown in FIG. 14.

Thereafter, when actuator button 80 is released, springs 62,92 act to push actuator button 80 and plunger 60 outward, thereby rotating trigger arm 150 counter-clockwise. As trigger arm 150 moves, spring 148 acts to urge primary and secondary pivot links 110,130 to rotate so as to move point B back to the right of the theoretical line between pivot points A and C to thereby reset control mechanism 90′. Thus, releasing actuator button 80 allows the collapsible catch formed by pivot links 110,130 to re-extend to the configuration that that again resists movement of the trigger arm toward pump main body 52.

As can be appreciated, control mechanism 90′ serves a similar function as control mechanism 90, in that both allow for a consistent amount of energy to be stored in actuation spring 92, and to be suddenly released when actuator button 80 is depressed to beyond a certain point. Of course, other control mechanisms can be used, with control mechanism 90 and control mechanism 90′ being but two exemplary options.

The present invention can be carried out in other specific ways than those herein set forth without departing from the scope and essential characteristics of the invention. Further, the various aspects of the disclosed device and method can be used alone or in any combination, as is desired. The disclosed embodiments are, therefore, to be considered in all respects as illustrative and not restrictive, and all changes coming within the meaning and equivalency range of the appended claims are intended to be embraced therein.

What is claimed is:

1. A nasal drug delivery device comprising:
a housing having a spray port;
storage container housing a liquidous medicament;
a selectively actuable pump supported by said housing and operatively connecting said reservoir to said spray port; said pump having a plunger controlling operation thereof;
an externally accessible actuator moveably coupled to said housing;
a first elastic element disposed operatively between said plunger and said actuator;
wherein inward movement of said actuator to a first distance causes said elastic element to compress and thereby store energy; and
wherein inward movement of said actuator beyond said first distance causes said plunger to be depressed by the release of said energy stored in said elastic element to thereby force a portion of said medicament to be sprayed from said spray port.

2. The device of claim 1 further comprising a catch associated with said plunger; wherein said catch resists inward movement of said plunger in response to inward movement of said actuator to said first distance to thereby compress said elastic element; wherein said catch moves from a first position to a second position in response to said actuator moving inward beyond said first distance to thereby release said plunger for inward movement under bias of said elastic element.

3. The device of claim 1 further comprising first and second pivot elements pivotally connected and cooperating to resist inward movement of said plunger in response to inward movement of said actuator to said first distance to thereby compress said elastic element; wherein said first and second pivot elements rotate relative to one another from a first position to a second position in response to said actuator moving inward beyond said first distance to thereby release said plunger for inward movement under bias of said elastic element.

4. The device of claim 1 further comprising a second elastic element acting on said plunger in opposition to said first elastic element and providing a reset bias to said plunger.

5. The device of claim 1 wherein said spray port comprises a nozzle imparting vertical flow to the medicament dispensed from the device.

6. The device of claim 1 wherein the storage container is a flexible and collapsible storage chamber.

7. The device of claim 1 wherein a force necessary to move said actuator inward beyond said first point is approximately four pounds.

8. The device of claim 1 further comprising a spray plume issuing from said spray port, said spray plume having a particle size distribution with a Dv50 value of approximately 50 um or less.

9. A method of administering a medicament nasally to a user comprising:
providing a nasal delivery device, said nasal delivery device comprising:
a housing having a spray port;
storage container housing a liquidous medicament;
a selectively actuable pump supported by said housing and operatively connecting said reservoir to said spray port; said pump having a plunger controlling operation thereof;
an externally accessible actuator moveably coupled to said housing;
a first elastic element disposed operatively between said plunger and said actuator;
storing energy in said first elastic element by depressing said actuator to a first distance while resisting movement of said plunger; and
depressing said actuator beyond said first distance to thereby release said stored energy to depress said plunger to thereby cause delivery of a portion of said medicament into the nasal passages of a user.

10. The method of claim 9 wherein said delivery of a portion of said medicament into the nasal passages of a user comprises generating a spray having a vertical flow as it exits the spray port.

11. The method of claim 9 further comprising thereafter releasing said actuator and returning said actuator to a ready position under bias of a second elastic element.

12. The method of claim 9 further comprising generating a spray of medicament from said spray port having a particle size distribution with a Dv50 value of approximately 50 um or less in response to said depressing said actuator.

13. A nasal medicament delivery device, comprising:
a housing extending in a first direction from a distal end portion to a proximal end portion and comprising a spray port disposed proximate said proximal end portion;
a reservoir operative to hold liquid medicament;
a selectively actuable pump operatively connecting said reservoir to said spray port;
said spray port oriented to spray medicament in a proximal second direction; said second direction forming a non-zero acute angle with said first direction.

14. The device of claim 13 wherein said housing has an upper surface and a lower surface and further comprising an externally accessible actuator disposed proximate said lower surface and operatively connected to said pump, wherein said spray port is disposed proximate said upper surface.

15. The device of claim 13 wherein said housing comprises a plurality of finger indentions on an upper surface thereof.

16. The device of claim 13 wherein said angle is in the range of about 30° to about 75°.

17. The device of claim 13 further comprising an externally accessible actuator depressible relative to said housing in a third direction to actuate said pump and cause said medicament to be sprayed from said spray port; wherein a dot product of a second vector oriented in said second direction and a third vector oriented in said third direction is a non-zero positive value.

18. The device of claim 13 wherein said pump comprises a plunger controlling operation thereof; wherein said device further comprises an externally accessible actuator moveably coupled to said housing and a first elastic element disposed operatively between said plunger and said actuator; wherein inward movement of said actuator to a first distance causes said elastic element to compress and thereby store energy; wherein inward movement of said actuator beyond said first distance causes said plunger to be depressed by the release of said energy stored in said elastic element to thereby force a portion of said medicament to be sprayed from said spray port.

19. The device of claim 18 further comprising a spray plume issuing from said spray port, said spray plume having a particle size distribution with a Dv50 value of approximately 50 um or less.

20. A method of administering a medicament nasally to a user comprising:
providing a nasal delivery device comprising:
a housing comprising distal end portion and a proximal end portion and a comprising a spray port disposed proximate said proximal end portion
storage container housing a liquidous medicament;
a manually powered pump supported by said housing and operatively connecting said reservoir to said spray port;
spray port configured to be inserted in a human user's nose; and
an externally accessible actuator moveable relative to said housing to actuate said pump and cause said medicament to be sprayed from said spray port;
disposing said proximal end portion proximate the user's face and said distal end portion distal from the user's face; a forward direction defined as extending from said distal end portion toward said proximal end portion;
thereafter, delivering a portion of said medicament into the nasal passages of the user by generating a spray of medicament from said spray port in a spray direction in response to depression of said actuator; and
wherein a dot product of a first vector oriented in said forward direction and a second vector oriented in said spray direction is a non-zero positive value.

21. The method of claim 20 wherein said generating a spray comprises generating a spray having a vertical flow as it exits the spray port.

22. The method of claim 20 wherein said externally accessible actuator is moveable relative to said housing in a third direction to actuate said pump and cause said medicament to be sprayed from said spray port; the method further comprising depressing said actuator in said third direction to actuate said pump and cause said medicament to be sprayed from said spray port, wherein a dot product of said second vector and a third vector oriented in said third direction is a non-zero positive value.

23. The method of claim 20 wherein said spray of medicament from said spray port has a particle size distribution with a Dv50 value of approximately 50 um or less.

24. The method of claim 20 wherein said pump comprises a plunger controlling operation thereof; wherein said nasal delivery device further comprises a first elastic element disposed operatively between said plunger and said actuator; wherein said generating a spray of medicament from said spray port in a spray direction in response to depression of said actuator comprises:
storing energy in said first elastic element by depressing said actuator to a first distance while resisting movement of said plunger; and
depressing said actuator beyond said first distance to thereby release said stored energy to depress said plunger to thereby generate a spray for delivery of a portion of said medicament into the nasal passages of a user.

25. The method of claim 24 wherein said actuator is depressible relative to said housing in a third direction, wherein a dot product of said second vector and a third vector oriented in said third direction is a non-zero positive value; wherein depressing said actuator beyond said first distance comprises depressing said actuator in said third direction.
